(19) United States
(12) Patent Application Publication
(10) Pub. No.: US 2023/0069180 A1
(43) Pub. Date: Mar. 2, 2023

(54) ENDOSCOPE NOSEPIECE AND ENDOSCOPE NOSEPIECE DISPOSAL METHOD

(71) Applicant: FUJIFILM Corporation, Tokyo (JP)

(72) Inventors: Takumi DEJIMA, Kanagawa (JP); Tomohiro OHKI, Kanagawa (JP); Yutaka SENDA, Kanagawa (JP); Ryo ISHIKAWA, Kanagawa (JP)

(73) Assignee: FUJIFILM Corporation, Tokyo (JP)

(21) Appl. No.: 18/052,998

(22) Filed: Nov. 7, 2022

Related U.S. Application Data
(63) Continuation of application No. PCT/JP2021/017376, filed on May 6, 2021.

(30) Foreign Application Priority Data
May 8, 2020 (JP) 2020-082835

(51) Int. Cl. A61B 1/00; C09J 7/38

(52) U.S. Cl. CPC A61B 1/00148; C09J 7/38; C09J 2301/302

(57) ABSTRACT

An endoscope nosepiece includes a nosepiece body and a drape. The nosepiece body has a pipe line through which an insertion part is inserted. The drape is provided integrally with the nosepiece body and covers a head of a patient. The nosepiece body is integrally provided with a stopper portion. The drape is located on a side of the pipe line facing an outside of a body of the patient with respect to the stopper portion.

### CROSS-REFERENCE TO RELATED APPLICATIONS

This application is a Continuation of PCT International Application No. PCT/JP2021/017376 filed on 6 May 2021, which claims priority under 35 U.S.C § 119(a) to Japanese Patent Application No. 2020-082835 filed on 8 May 2020. The above application is hereby expressly incorporated by reference, in its entirety, into the present application.

### BACKGROUND OF THE INVENTION

### 1. Field of the Invention

The present invention relates to an endoscope nosepiece used by being worn on a nose of a patient in a case where an insertion part of an endoscope is inserted through the nose, and an endoscope nosepiece disposal method.

### 2. Description of the Related Art

In recent years, it has been possible to make the diameter of an insertion part of an endoscope extremely small (for example, to make the diameter 6 mm or less), and nasal type endoscopes in which the insertion part is inserted through an external nostril have been put into practical use. Medical examinations using the nasal type endoscopes have been becoming rapidly widespread because vomiting caused by a pharyngeal reflex is less likely to occur and pain suffered by a patient is less than that of oral type endoscopes in which the insertion part is inserted through a mouth.

Since a mucous membrane of an inner wall of the nose is weaker than that of the mouth, in a case where the mucous membrane comes into direct contact with a nasal type endoscope and they rub against each other, nasal bleeding is likely to occur. Thus, it is common to use an endoscope nosepiece to be inserted into the external nostril. This endoscope nosepiece has a pipe line through which the insertion part of the endoscope is inserted, the insertion part of the endoscope can be easily introduced into a body through the pipe line, and the mucous membrane of the nose can be protected (see, for example, JP2009-125345A).

### SUMMARY OF THE INVENTION

In the medical field, it is desired to take further preventive measures against infectious diseases. In particular, it is important to take measures to prevent droplet infection caused by inhaling droplets caused by coughing or sneezing of infectious disease patients.

However, in a case where the endoscope nosepiece as in JP2009-125345A is used, the nose of the patient is closed by the endoscope nosepiece, but the mouth of the patient is exposed. Thus, in a case where the patient coughs or sneezes in a case where the insertion part of the endoscope is inserted through the nose using the endoscope nosepiece, there is a possibility that the droplets discharged from the mouth of the patient are inhaled by persons around the patient.

An object of the present invention is to provide an endoscope nosepiece and an endoscope nosepiece disposal method that can prevent diffusion of droplets discharged from a mouth of a subject in a case where an insertion part of an endoscope is inserted through the nose.

An endoscope nosepiece according to an aspect of the present invention comprises a nosepiece body and a drape. The nosepiece body is a nosepiece body that is inserted into an external nostril of a subject in a case where an insertion part of an endoscope is inserted into the subject through a nose and that has a pipe line through which the insertion part is inserted. The drape protrudes from an outer peripheral edge of the nosepiece body and is provided integrally with the nosepiece body to cover at least a part of the subject.

It is preferable that the drape is formed in a shape of a sheet and is in a folded state when not in use, and a holding member is stuck on the drape in the folded state to hold the drape in the folded state. The holding member is at least one of a pressure-sensitive adhesive tape, a locking member, a constraint member, or a coating member.

It is preferable that a mouthpiece is held by a mouth of the subject and is provided integrally with the drape, and the drape protrudes from an outer peripheral edge of the mouthpiece.

It is preferable that the nosepiece body has an opening portion provided at an end of the pipe line on a side located outside a body of the subject in a case where the nosepiece body is inserted into the external nostril of the subject, and a fluid suppression member is provided in the opening portion to suppress passage of a fluid in the pipe line.

It is preferable that the fluid suppression member has a first porous member having a first slit parallel to an insertion direction of the insertion part formed therein, and a second porous member having a second slit parallel to the insertion direction of the insertion part and intersecting the first slit formed therein, and is attached to the opening portion.

It is preferable that the fluid suppression member has a first porous member having at least three first slits formed therein, and a second porous member having at least three second slits formed therein, and is attached to the opening portion.

It is preferable that three first slits are formed in the first porous member, and three second slits are formed in the second porous member.

It is preferable that the first porous member and the second porous member have a rectangular outer shape, one of the first slits is disposed to be perpendicular to one side of the first porous member, one of the second slits is disposed to be perpendicular to one side of the second porous member, and the first porous member and the second porous member are attached to the opening portion in a state in which the second slit is disposed at a position rotated by 180° around a central axis parallel to the insertion direction with respect to the first slit. It is preferable that the fluid suppression member is attached to the opening portion in a state where the first porous member and the second porous member are laminated.

An endoscope nosepiece disposal method according to an aspect of the present invention is an endoscope nosepiece disposal method using an endoscope nosepiece including a nosepiece body that is inserted into an external nostril of a subject in a case where an insertion part of an endoscope is inserted into the subject through a nose, and a drape that is provided integrally with the nosepiece body and that covers at least a part of the subject. The disposal method comprises discarding the endoscope nosepiece by removing the insertion part from the subject and then removing the endoscope nosepiece from the nose of the subject together with the drape.

An endoscope nosepiece disposal method according to an aspect of the present invention is an endoscope nosepiece disposal method using an endoscope nosepiece that is inserted into an external nostril of a subject in a case where an insertion part of an endoscope is inserted into the subject through a nose, and a drape that is provided separately from the endoscope nosepiece and that covers at least a part of the subject. The disposal method comprises discarding the endoscope nosepiece by removing the insertion part from the subject and then removing the endoscope nosepiece from the nose of the subject together with the drape.

According to the present invention, in a case where the insertion part of the endoscope is inserted through the nose, it is possible to prevent the diffusion of droplets discharged from the mouth of the subject.

### BRIEF DESCRIPTION OF THE DRAWINGS

FIG. 1 is a schematic view showing endoscopy using a nasal type endoscope.

FIG. 2 is a perspective view of an endoscope nosepiece.

FIG. 3 is a perspective view of the endoscope nosepiece in an unused state.

FIG. 4 is an exploded perspective view showing a configuration of the endoscope nosepiece.

FIG. 5 is a cross-sectional view showing the configuration of the endoscope nosepiece.

FIG. 6 is a cross-sectional view of major parts of the endoscope nosepiece.

FIGS. 7A and 7B are explanatory views illustrating a method of folding a drape in a front-back direction.

FIGS. 8A and 8B are explanatory views illustrating the method of folding a drape in a longitudinal direction.

FIG. 9 is an explanatory view illustrating an operation in a case where the endoscope nosepiece is worn on a nose of a patient.

FIG. 10 is an explanatory view showing a state in which the endoscope nosepiece is worn on the nose.

FIG. 11 is a schematic view showing endoscopy in a second embodiment.

FIG. 12 is a perspective view of an endoscope nosepiece according to the second embodiment.

FIG. 13 is an exploded perspective view of the endoscope nosepiece according to the second embodiment.

FIG. 14 is a cross-sectional view of major parts of the endoscope nosepiece according to the second embodiment.

FIG. 15 is an exploded perspective view of an endoscope nosepiece according to a third embodiment.

FIG. 16 is a perspective view showing a configuration of a fluid suppression member.

FIG. 17 is an explanatory view illustrating a state in which an insertion part is inserted into the fluid suppression member.

FIG. 18 is an exploded perspective view of a first modification example of the endoscope nosepiece.

FIG. 19 is a perspective view showing a configuration of a fluid suppression member in the first modification example.

FIG. 20 is a front view of a second modification example of the endoscope nosepiece.

FIG. 21 is an exploded perspective view showing a configuration of a drape adapter in a second modification example.

FIG. 22 is an explanatory view illustrating an operation of attaching an end part of a fixing band to a band attachment part.

FIG. 23 is an explanatory view showing a state in which an endoscope nosepiece in a third modification example is worn on the nose.

### DESCRIPTION OF THE PREFERRED EMBODIMENTS

### First Embodiment

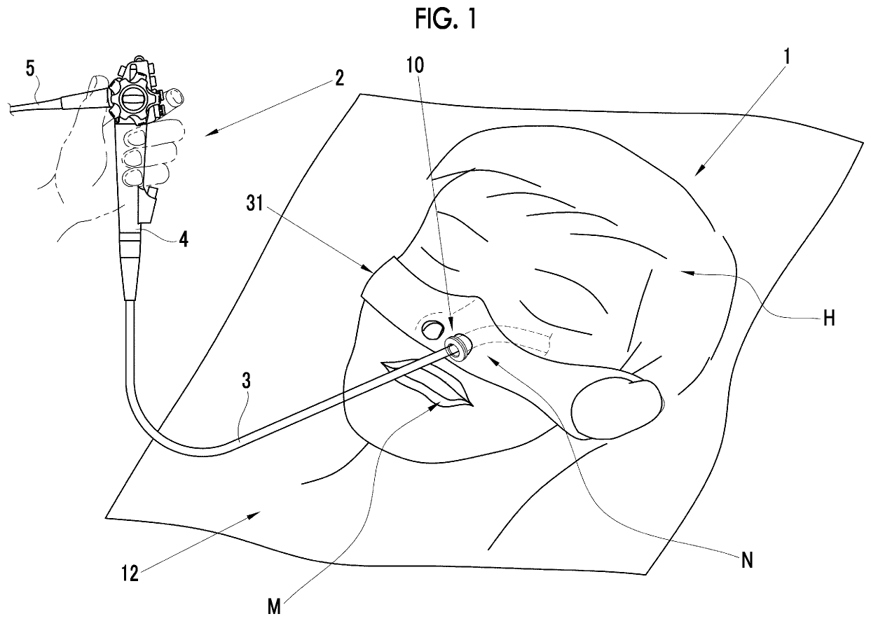

As shown in FIG. 1, an endoscope nosepiece 10 of the embodiment of the present invention is used for endoscopy using a nasal type endoscope 2. The endoscope 2 is, for example, an upper gastrointestinal endoscope, and comprises an insertion part 3 inserted into an upper gastrointestinal tract of a patient 1 who is a subject, an operating part 4 consecutively installed at a proximal end part of the insertion part 3, and a universal cord 5 connected to the operating part 4. The universal cord 5 is connected to an external device such as a processor device or a light source device via a connector (not shown).

Although not shown, a distal end surface of the insertion part 3 is provided with an observation window and an illumination window. An image sensor (not shown) is disposed behind the observation window, and an optical fiber cable (not shown) is disposed behind the illumination window. A signal line of the image sensor and the optical fiber cable are connected to a processor device and a light source device, respectively, through the insertion part 3, the operating part 4, and the universal cord 5. The processor device performs image processing or the like on an endoscopic image captured by the image sensor and displays the image-processed image on a monitor.

In a case where the insertion part 3 of the endoscope 2 is inserted through a nose N of the patient 1, the endoscope nosepiece 10 is worn on the nose N of the patient 1.

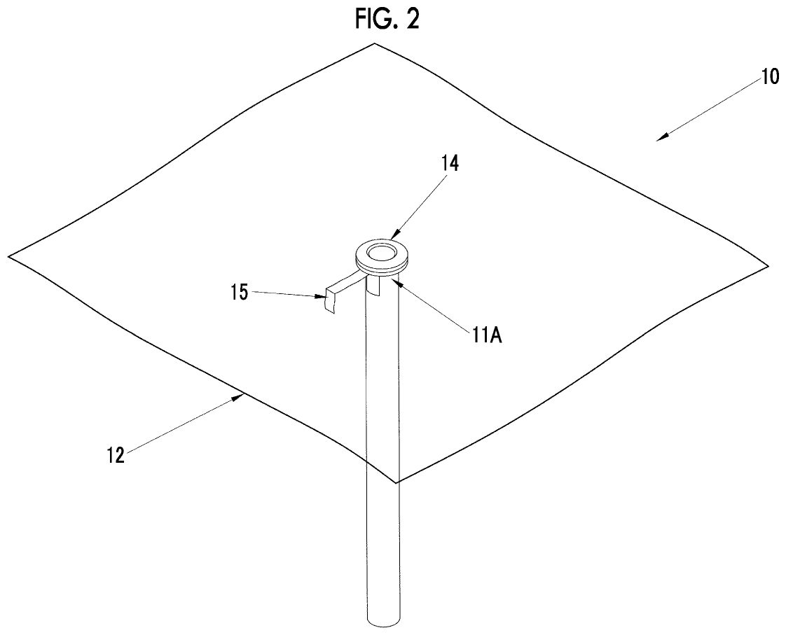

As shown in FIG. 2, the endoscope nosepiece 10 includes a nosepiece body 11A, a drape 12, a retaining member 14, and a pressure-sensitive adhesive tape 15. The drape 12 has a small thickness and is formed in the shape of a quadrangular sheet, for example, is formed of a transparent vinyl sheet. The drape 12 has an area that covers and hides at least a part of the subject, for example, a head H of the patient 1 (see FIG. 1).

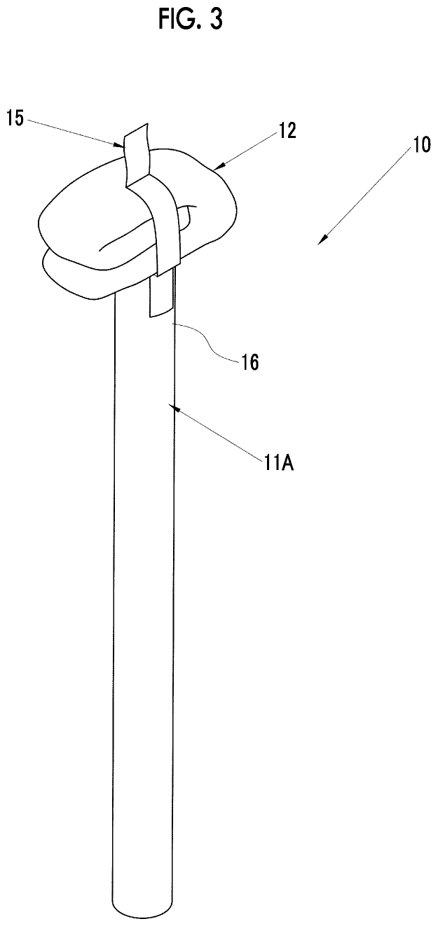

As shown in FIG. 3, the drape 12 is in a folded state when not in use and has the pressure-sensitive adhesive tape 15 stuck on an end part thereof. By applying the pressure-sensitive adhesive tape 15, the drape 12 is held in the folded state. In addition, the method of folding the drape 12 and the method of applying the pressure-sensitive adhesive tape 15 will be described below.

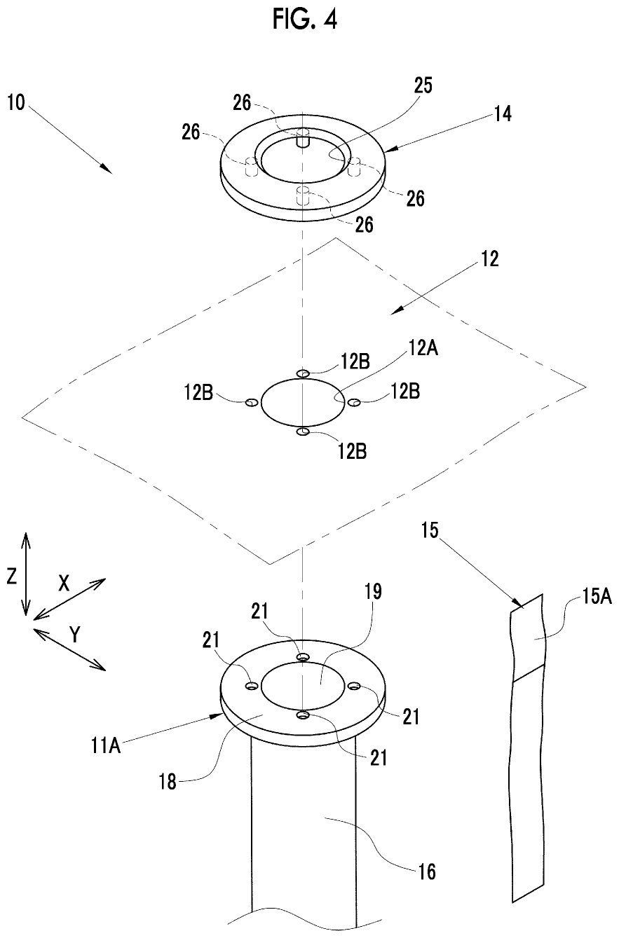

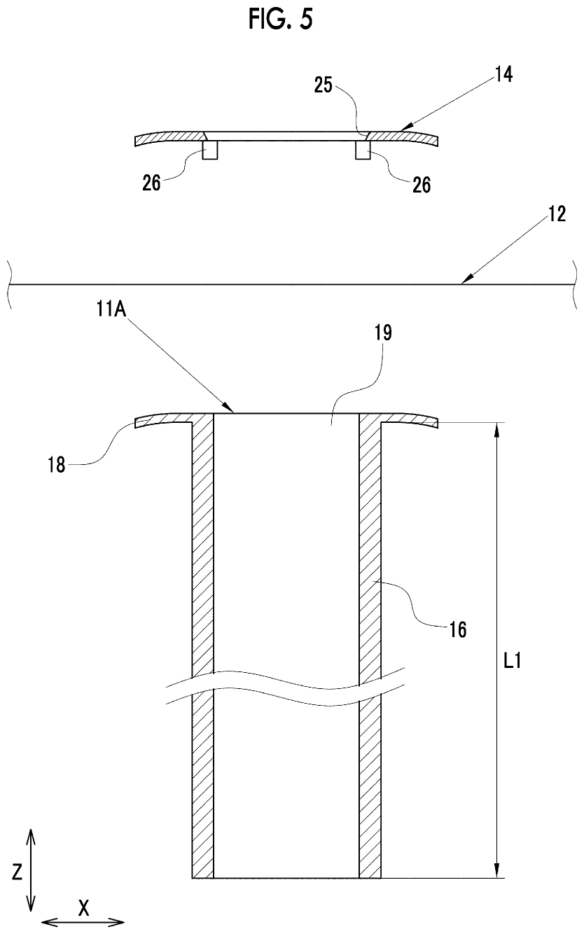

As shown in FIGS. 4 and 5, the nosepiece body 11A is integrally provided with a tubular portion 16 and a stopper portion 18. The nosepiece body 11A is formed of, for example, a soft material such as a soft resin to be easily worn on the nose N of the patient 1. The tubular portion 16 is inserted into the nose N of the patient 1 in a case where the insertion part 3 of the endoscope 2 is inserted into the patient 1 through the nose. The tubular portion 16 is formed to have, for example, a circular or elliptical cross section such that the tubular portion 16 can be easily inserted into the nose N. It is preferable that a length L1 of the tubular portion 16 may be, for example, a dimension within a range longer than a path length from the external nostril to the most constricted portion of a middle nasal meatus and shorter than a path length from the external nostril to an upper pharynx.

Inside the tubular portion 16, there is a pipe line 19 through which the insertion part 3 is inserted. The pipe line 19 is a through-hole having a circular or elliptical cross section. An inner diameter of the pipe line 19 is formed in conformity with an outer diameter of the insertion part 3. The tubular portion 16 has a plurality of fitting holes 21 provided at the end located outside a body of the patient 1 in a case where the tubular portion 16 is inserted into the nose N. Each fitting hole 21 is disposed around the pipe line 19 and has a fitting pin 26 described below fitted thereinto.

The stopper portion 18 is formed in a disk shape that protrudes from an outer diameter of the tubular portion 16. An outer diameter of the stopper portion 18 is larger than a diameter of the external nostril of the nose N. Accordingly, the stopper portion 18 can be stuck on the external nostril to prevent the tubular portion 16 from being immersed, and the stopper portion 18 can cover and hide the external nostril. The thickness of the stopper portion 18 is formed to be larger than, for example, the wall thickness of the pipe line 19. Accordingly, even in a case where the pipe line 19 is pulled into a nasal cavity of the nose N, the stiffness is secured such that the stopper portion 18 is not pulled into the external nostril. Additionally, the stopper portion 18 also serves as a handle in a case where the nosepiece body 11A is inserted into the nose N of the patient 1.

The retaining member 14 is formed in a thin plate shape. Specifically, the outer shape of the retaining member 14 is formed in a disk shape that matches the stopper portion 18. The retaining member 14 is formed of, for example, a soft material such as a soft resin.

The retaining member 14 is formed with an opening portion 25, and four fitting pins 26 (see FIG. 5). The opening portion 25 is disposed at a position facing the pipe line 19 of the nosepiece body 11A. Each fitting pin 26 protrudes from a position around the opening portion 25 and facing the pipe line 19. The fitting pin 26 fits into the fitting hole 21 of the nosepiece body 11A. Accordingly, the nosepiece body 11A and the retaining member 14 are combined with each other.

As shown in FIG. 4, the drape 12 is formed with an opening portion 12A that matches the pipe line 19 and an opening portion 12B that matches the fitting hole 21. Since the drape 12 does not block the pipe line 19 by forming the opening portion 12B, the insertion part 3 can be inserted into the pipe line 19. In addition, for convenience of illustration, the drape 12 in FIG. 4 is shown by cutting out only the periphery of the opening portion 12A (a range surrounded by a two-dot chain line), and the actual size thereof is larger than the range surrounded by the two-dot chain line.

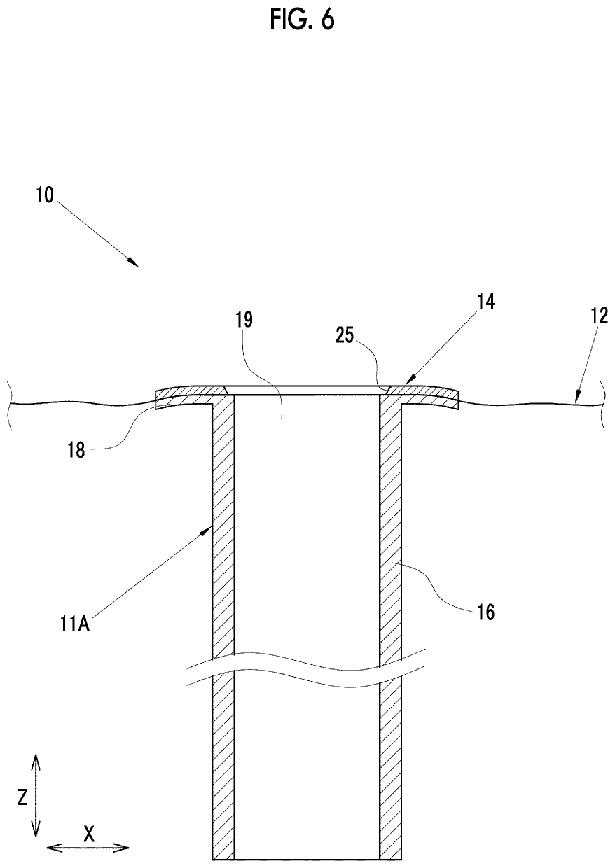

As shown in FIG. 6, the retaining member 14 sandwiches the drape 12 with the nosepiece body 11A. In this case, the retaining member 14 is anchored to the nosepiece body 11A by fitting the fitting pin 26 of the retaining member 14 into the opening portion 12B of the drape 12 and the fitting hole 21 of the nosepiece body 11A.

As described above, the drape 12 is sandwiched and fixed between the nosepiece body 11A and the retaining member 14. Accordingly, in a case where the tubular portion 16 is inserted into the nose N, the drape 12 is located on the side facing the outside of the body of the patient 1 with respect to the stopper portion 18.

The drape 12 sandwiched between the nosepiece body 11A and the retaining member 14 protrudes from an outer peripheral edge of the nosepiece body 11A and is provided integrally with the nosepiece body 11A (a state shown in FIG. 2), while the drape 12 is in the folded state when the endoscope nosepiece 10 is not in use, and the folded state is maintained by the pressure-sensitive adhesive tape 15 (a state shown in FIG. 3). A method of folding the drape 12 and a method of applying the pressure-sensitive adhesive tape 15 will be described below.

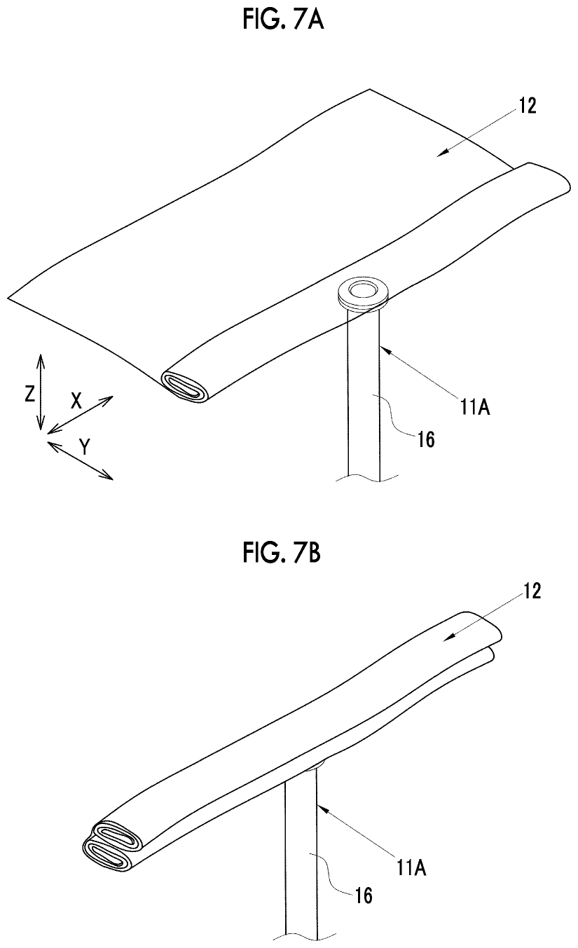

As shown in FIG. 7A, in a case where the drape 12 is folded, for example, the drape 12 is rolled from one end in a front-back direction Y, and the drape 12 is rolled to the position of the nosepiece body 11A located at the center, and then, the drape 12 is also rolled from the other end in the front-back direction Y. Accordingly, as shown in FIG. 7B, the drape 12 has an elongated belt shape.

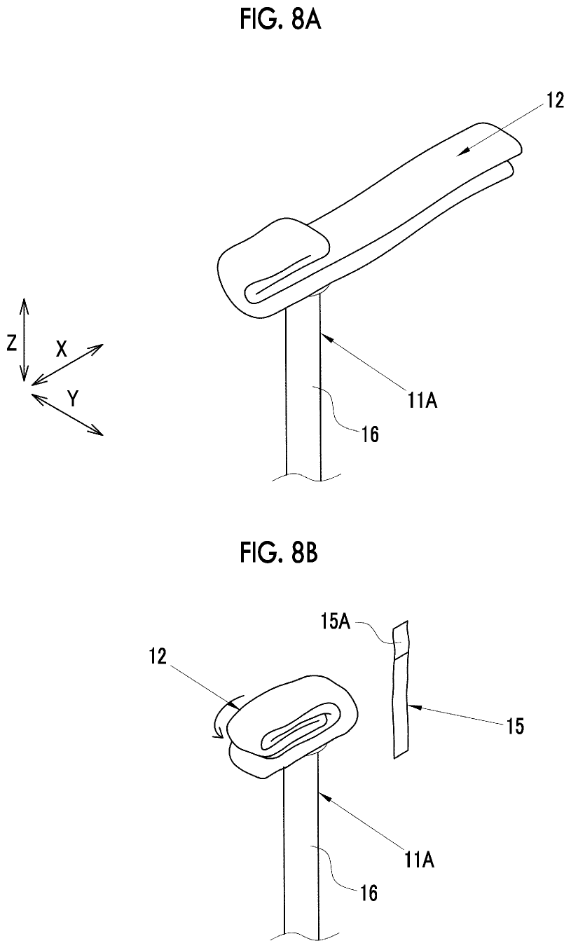

Next, as shown in FIG. 8A, the drape 12 is folded to be rolled from one end having the elongated belt shape. Then, after the drape 12 is rolled to the position of the nosepiece body 11A located at the center, the drape 12 is also rolled from the other end having the elongated belt shape. Accordingly, as shown in FIG. 8B, the drape 12 is in a small folded state. Then, the pressure-sensitive adhesive tape 15 is stuck on an end part of the drape 12 from the nosepiece body 11A. An end part 15A of the pressure-sensitive adhesive tape 15 does not adhere to the drape 12 but is made to protrude from the outer shape of the drape 12. In this case, for example, the end part 15A is folded back, and the pressure-sensitive adhesive surfaces thereof are stuck to each other in advance to form a portion where the pressure-sensitive adhesive surfaces are not exposed, on the pressure-sensitive adhesive tape 15.

By applying the pressure-sensitive adhesive tape 15 as described above, the folded state of the drape 12 is maintained (the state shown in FIG. 3). Additionally, by making the end part 15A not adhere to the drape 12, in a case where the endoscope nosepiece 10 is used, it is easy to grip the end part 15A and to peel off the pressure-sensitive adhesive tape 15.

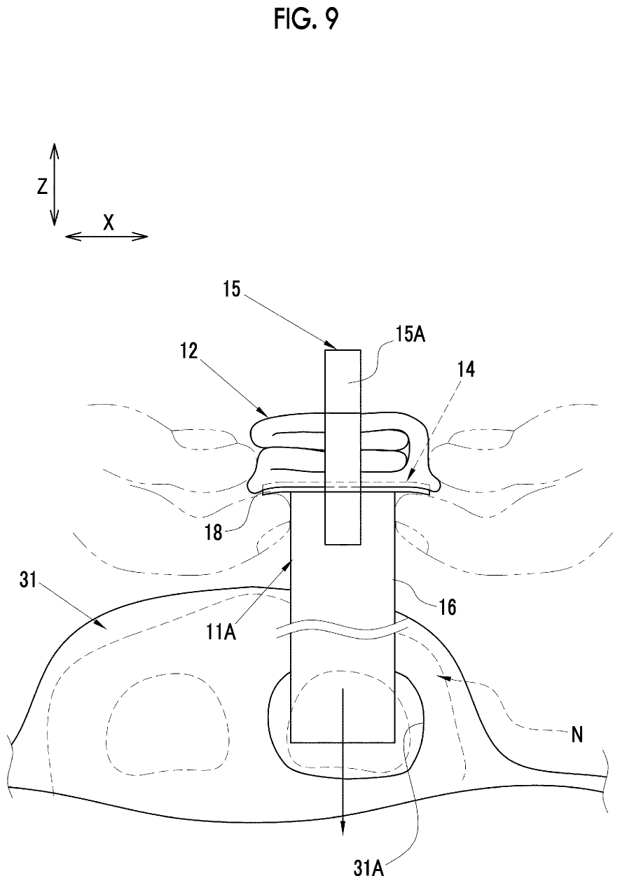

The operation in a case where a doctor, who is a user, makes the endoscope nosepiece 10 be worn on the nose N of the patient 1 and inserts the insertion part 3 through the pipe line 19 will be described. As shown in FIG. 9, first, the doctor makes a nose mask 31 be worn on the patient 1. The nose mask 31 covers a major portion of the nose N of patient 1. The nose mask 31 is formed with a substantially circular opening 31A at a position corresponding to one of the external nostrils of the nose N. In addition, although not limited to this, a nose mask of a type that covers the nose N and a mouth M of the patient 1 may be worn.

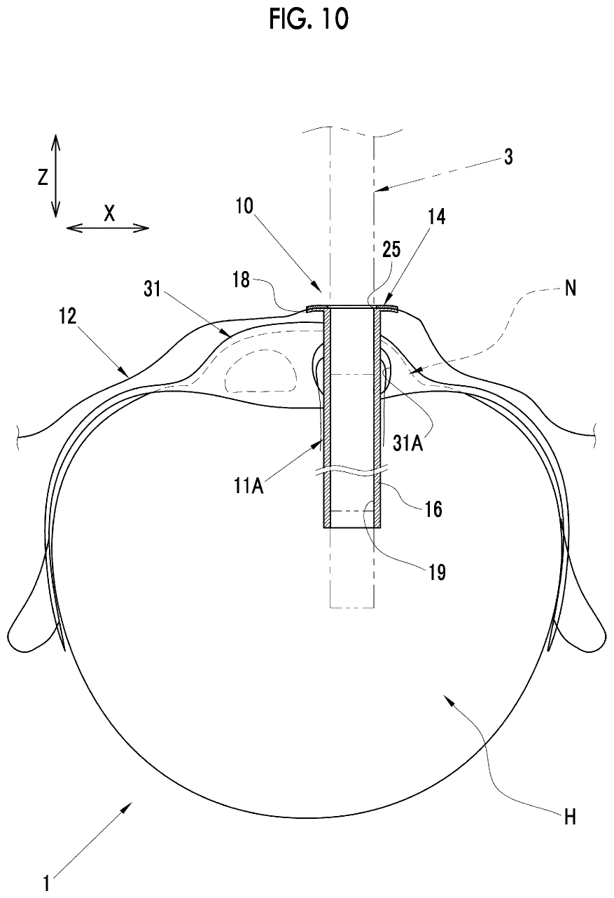

After the nose mask 31 is worn on the patient 1, the doctor grips the stopper portion 18 and the tubular portion 16 and inserts the tubular portion 16 into the external nostril of the nose N of the patient 1 through the opening 31A. The tubular portion 16 is inserted to a position where the stopper portion 18 comes into contact with the nose mask 31. Accordingly, the endoscope nosepiece 10 is worn on the nose N. After mounting the endoscope nosepiece 10 on the nose N, the doctor peels the pressure-sensitive adhesive tape 15 off from the nosepiece body 11A and the drape 12. By peeling off the pressure-sensitive adhesive tape 15, the drape 12 can be brought to an original state, that is, a spread state thereof from the folded state. Then, as shown in FIG. 10, by mounting the endoscope nosepiece 10 on the nose N and bringing the drape 12 into the spread state, the head H of the patient 1 can be covered and hidden with the drape 12.

As described above, after the endoscope nosepiece 10 is mounted on the nose N and the head H of the patient 1 is covered and hidden with the drape 12, the doctor inserts the insertion part 3 of the endoscope 2 through the pipe line 19 of the endoscope nosepiece 10 into the body of the patient 1 in an insertion direction Z through the nose N. Even in a case where the patient 1 coughs or sneezes in a case where the insertion part 3 is inserted through the nose N, the drape 12 covers and hides the head H of the patient 1. Therefore, diffusion of the droplets discharged from the mouth M (see FIG. 1) of the patient 1 can be prevented by the drape 12. Thus, since inhalation of the droplets discharged from the mouth M of the patient 1 by a person around the patient can be suppressed, droplet infection can be reliably prevented.

The endoscope nosepiece 10 is a disposable type in which the insertion part 3 is removed from the nose N of the patient 1, and is then removed from the nose N of the patient 1 together with the drape 12 and discarded. Accordingly, since the endoscope nosepiece 10 can be removed from the nose of the patient 1 and then be wrapped in the drape 12 and discarded, the adhering droplets of the patient 1 are not diffused, and the endoscope nosepiece 10 can be discarded.

### Second Embodiment

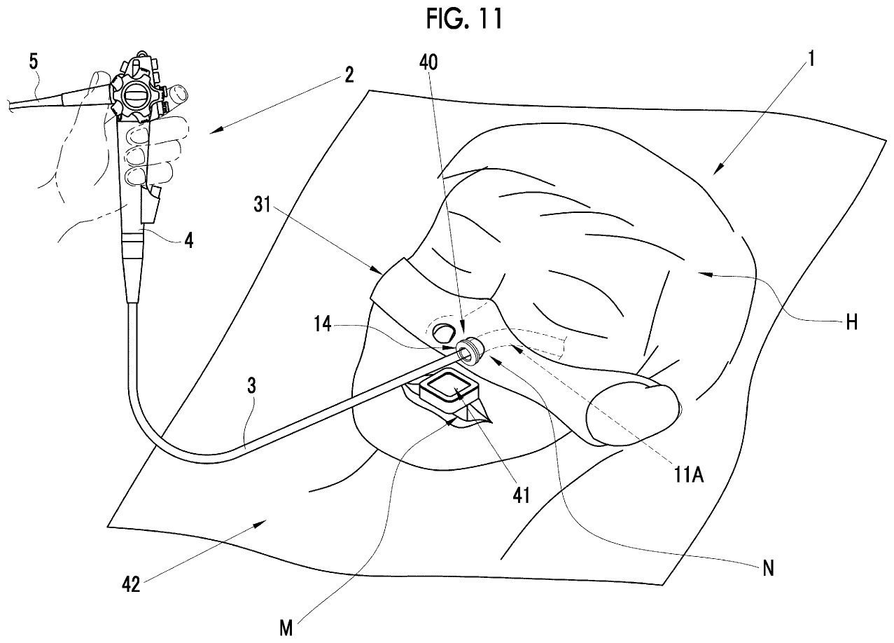

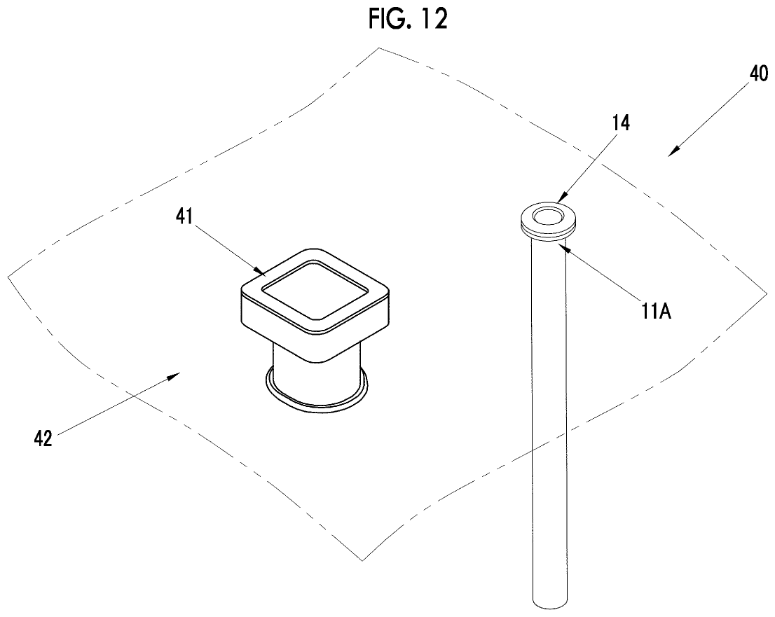

In the second embodiment, in addition to the configuration of the endoscope nosepiece of the above first embodiment, a configuration comprising a mouthpiece will be described. As shown in FIGS. 11 and 12, an endoscope nosepiece 40 includes a mouthpiece 41, a nosepiece body 11A, a drape 42, and a retaining member 14. In addition, the nosepiece body 11A and the retaining member 14 have the same configuration as that of the endoscope nosepiece 10 of the above first embodiment, and the same components and the like will be designated by the same reference numerals, and the description thereof will be omitted.

Similarly to the drape 12 of the above first embodiment, the drape 42 has a small thickness and is formed in the shape of a quadrangular sheet, for example, is formed of a transparent vinyl sheet. The drape 42 has an area that covers and hides at least a part of the subject, for example, the head H of the patient 1.

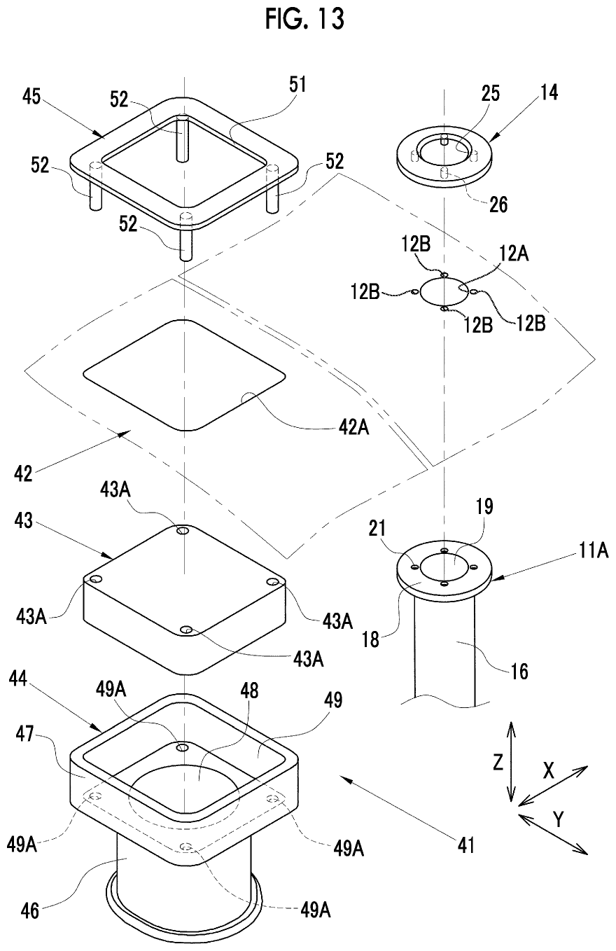

As shown in FIG. 13, the drape 42 has an opening portion 42A formed in addition to the opening portion 12A and the opening portion 12B similar to the drape 12 of the above first embodiment. The opening portion 12A has a shape that matches the outer shape of a porous member 43 described below. Accordingly, since the drape 42 does not block the porous member 43, the patient 1 can breathe through the porous member 43. In addition, for convenience of illustration, the drapes 42 in FIGS. 12 and 13 are shown by cutting out only the peripheries of the opening portion 12A, the opening portion 12B, and the opening portion 42A (a range surrounded by a two-dot chain line), and the actual size thereof is larger than the range surrounded by the two-dot chain line.

Similar to the above first embodiment, the retaining member 14 sandwiches the drape 42 with the nosepiece body 11A. The drape 42 is sandwiched and fixed between the nosepiece body 11A and the retaining member 14.

The mouthpiece 41 includes the porous member 43, a mouthpiece body 44, and a retaining member 45. The mouthpiece body 44 is integrally provided with a tubular portion 46 and a frame portion 47. The mouthpiece body 44 is formed of, for example, a soft material such as a soft resin to be easily mounted on the mouth M of the patient 1. The tubular portion 46 is a portion held by the mouth M of the patient 1. The tubular portion 46 is formed in, for example, an elliptical cross-sectional shape.

The frame portion 47 is consecutively installed at the tubular portion 46 and is formed in a quadrangular frame shape. The frame portion 47 is located outside the body of the patient 1 in a case where the tubular portion 46 is held by the mouth M. A pipe line 48 through which air passes is provided inside the tubular portion 46 and the frame portion 47. The pipe line 48 is a through-hole having a circular or elliptical cross section.

The pipe line 48 has an opening portion 49 provided at the end on a side located outside the body of the patient 1 in a case where the tubular portion 46 is held by the mouth M. The opening portion 49 is a rectangular opening portion that matches the frame portion 47. Fitting holes 49A into which fitting pins 52, which will be described below, are fitted are formed at the four corners of the opening portion 49.

As the porous member 43, a porous member is used which is fine enough such that air necessary for breathing is allowed to pass therethrough and liquids such as droplets are blocked. The porous member 43 is formed in the shape of a rectangle or a rectangular plate and is formed with a size to be housed in the opening portion 49. Additionally, the porous member 43 has fitting holes 43A formed at positions near the four corners. The fitting holes 43A are disposed at positions corresponding to the fitting holes 49A of the mouthpiece body 44.

The retaining member 45 is formed in a thin plate shape. The outer shape of the retaining member 45 is a shape that matches the frame portion 47. The retaining member 14 is formed of, for example, a soft material such as a soft resin.

The retaining member 45 is formed with an opening portion 51, and four fitting pins 52 (see FIG. 13). The opening portion 51 is disposed at a position facing the opening portion 49 of the mouthpiece body 44. Each fitting pin 52 protrudes from a position around the opening portion 51 and facing the opening portion 49. The fitting pin 52 fits into the fitting hole 49A of the mouthpiece body 44. Accordingly, the mouthpiece body 44 and the retaining member 45 are combined with each other.

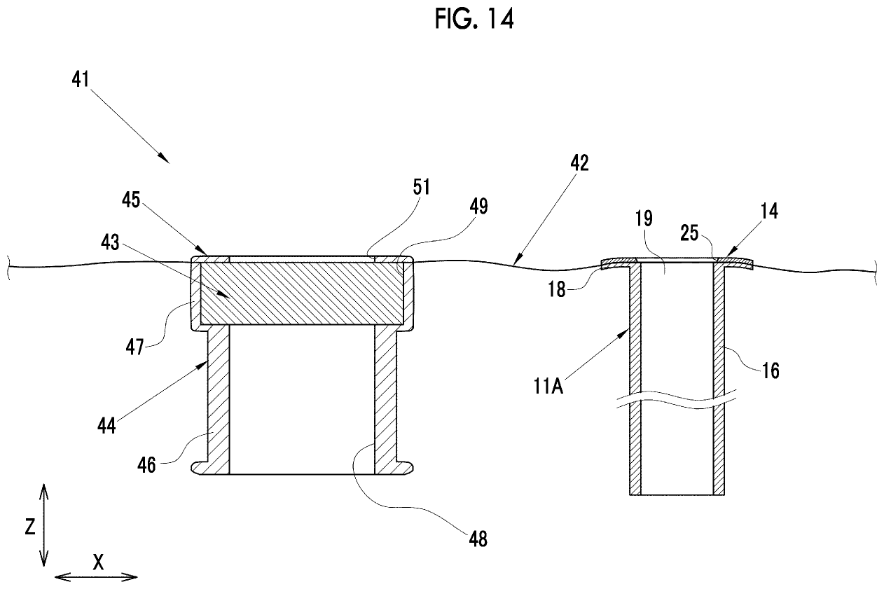

As shown in FIG. 14, the retaining member 45 sandwiches the drape 42 with the mouthpiece body 44 in a state in which the porous member 43 is housed in the opening portion 49. In this case, by fitting the fitting pin 52 of the retaining member 45 into the fitting hole 49A of the porous member 43 and the fitting hole 43A of the mouthpiece body 44, the retaining member 45 can be anchored to the mouthpiece body 44. Since the position of the porous member 43 is restricted by the retaining member 45, the porous member 43 does not separate from the opening portion 49.

As described above, the drape 42 sandwiched between the mouthpiece body 44 and the retaining member 45 protrudes from an outer peripheral edge of the mouthpiece 41 and is provided integrally with the mouthpiece 41 (a state shown in FIG. 12). In addition, similar to the drape 12 of the above first embodiment, it is preferable that the drape 42 is in the folded state when the endoscope nosepiece 40 is not in use, and the folded state is maintained by the pressure-sensitive adhesive tape 15.

After the nosepiece body 11A is worn on the nose N and the mouthpiece body 44 is worn on the mouth M, the doctor peels off the pressure-sensitive adhesive tape 15. By peeling off the pressure-sensitive adhesive tape 15, the drape 42 can be brought to a spread state thereof from the folded state. Then, by mounting the nosepiece body 11A on the nose N, mounting the mouthpiece body 44 on the mouth M, and bringing the drape 42 into the spread state, the head H of the patient 1 can be covered and hidden with the drape 12.

As described above, after the nosepiece body 11A is mounted on the nose N, the mouthpiece body 44 is mounted on the mouth M, and the head H of the patient 1 is covered and hidden with the drape 42, the doctor inserts the insertion part 3 of the endoscope 2 through the pipe line 19 of the endoscope nosepiece 40 into the body of the patient 1 in the insertion direction Z through the nose N. Similar to the endoscope nosepiece 10 of the above first embodiment, since the drape 42 covers and hides the head H of the patient 1, the drape 42 can prevent the diffusion of the droplets discharged from the mouth M of the patient 1. Additionally, since the mouthpiece 41 capable of passing air is worn on the mouth M of the patient 1, the breathing of the patient 1 is not hindered.

Additionally, similar to the above first embodiment, the endoscope nosepiece 10 can be removed from the nose of the patient 1 and then be wrapped in the drape 42 and discarded. Therefore, the adhering droplets of the patient 1 can be discarded without being diffused.

In addition, in the above second embodiment, the mouthpiece 41 is made of the porous member 43, the mouthpiece body 44, and the retaining member 45. However, the mouthpiece 41 is not limited to this, and the entire mouthpiece 41 may be made of a sponge member.

### Third Embodiment

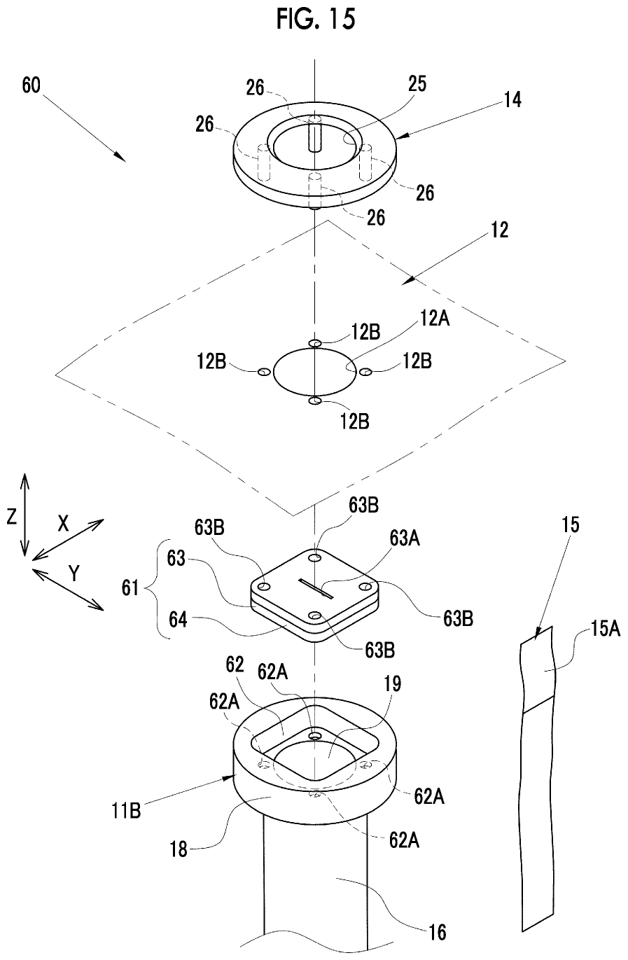

In the third embodiment, a configuration comprising a fluid suppression member in addition to the configurations of the above first and second embodiments will be described. As shown in FIG. 15, an endoscope nosepiece 60 includes a nosepiece body 11B, a drape 12, a retaining member 14, and a fluid suppression member 61. In addition, the retaining member 14 has the same configuration as that of the endoscope nosepiece 10 of the above first embodiment, and the same components and the like will be designated by the same reference numerals, and the description thereof will be omitted.

In the nosepiece body 11B of the present embodiment, the pipe line 19 has an opening portion 62 provided at an end on a side located outside the body of the patient 1 in a case where the tubular portion 16 is inserted into the external nostril of the nose N. In addition, the nosepiece body 11B is the same as the nosepiece body 11A of the above first embodiment except that the nosepiece body 11B has the opening portion 62. The opening portion 62 is a rectangular opening portion. Fitting holes 62A into which the fitting pins 26 are fitted are formed at the four corners of the opening portion 62.

The opening portion 62 is provided with the fluid suppression member 61. In a case where the tubular portion 16 is inserted into the external nostril of the nose N, the fluid suppression member 61 suppresses the passage of a fluid in the pipe line 19 leading to the inside of the body of the patient 1. Specifically, the fluid suppression member 61 allows gas such as air necessary for breathing to pass through and blocks liquid such as droplets. In addition, an axial dimension of the stopper portion 18 in the present embodiment is larger than the stopper portions 18 in the above first and second embodiments by the size of the fluid suppression member 61 housed therein.

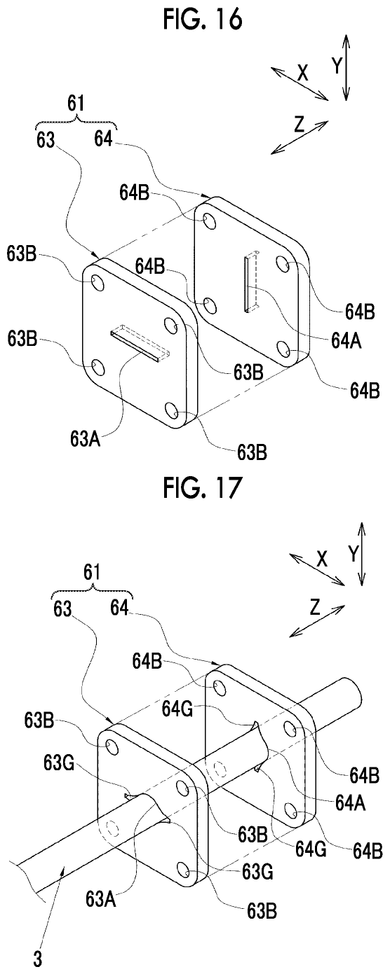

As shown in FIG. 16, the fluid suppression member 61 has a first porous member 63 and a second porous member 64. The first porous member 63 is formed by forming a flexible porous material into a rectangular plate shape. The first porous member 63 has a first slit 63A. The first porous member 63 is formed with a size such that the first porous member is housed in the opening portion 62. The first slit 63A is formed parallel to a left-right direction X and parallel to the insertion direction Z of the insertion part 3. The left-right direction X is a direction perpendicular to the insertion direction Z. Additionally, in the first porous member 63, fitting holes 63B are formed at positions near the four corners and at positions where the first porous member 63 does not interfere with the first slit 63A. The fitting holes 63B are disposed at positions corresponding to the fitting holes 62A of the nosepiece body 11B.

The second porous member 64 is formed by forming a flexible porous material into a disk shape. The second porous member 64 has a second slit 64A. The second porous member 64 is formed with a size such that the second porous member is housed in the opening portion 62. The second slit 64A is formed in a direction parallel to the insertion direction Z and intersecting the first slit 63A. More specifically, the second slit 64A is formed parallel to the insertion direction Z and parallel to the front-back direction Y. The front-back direction Y is a direction perpendicular to the left-right direction X and to the insertion direction Z.

The porous material that forms the first porous member 63 and the second porous member 64 is a porous material having a pore diameter and a structure that allows gas such as air necessary for breathing to pass therethrough and that blocks liquids such as droplets. For example, a synthetic sponge obtained by foam-molding a resin or a natural sponge such as a sponge is used. The first porous member 63 and the second porous member 64 may be formed separately, and the first slit 63A and the second slit 64A may be made to adhere to each other and be integrated with each other in an intersecting state.

In the fluid suppression member 61, the first porous member 63 and the second porous member 64 are housed in the opening portion 62, and the positions thereof in the insertion direction Z are restricted by the retaining member 14. Accordingly, the first porous member 63 and the second porous member 64 are attached to the opening portion 62 in a laminated state.

As shown in FIG. 17, in a case where the insertion part 3 is inserted into the pipe line 19 through the fluid suppression member 61, the insertion part 3 moves in the insertion direction Z while an outer peripheral surface of the insertion part 3 comes into close contact with the first slit 63A and the second slit 64A. In this case, gaps 63G and 64G are formed at both end parts of the first slit 63A and the second slit 64A. However, since the first slit 63A and the second slit 64A intersect each other as described above, the positions of the gaps 63G and 64G do not overlap each other.

The retaining member 14 sandwiches the drape 12 with the nosepiece body 11B in a state in which the fluid suppression member 61 is housed in the opening portion 62. In this case, by fitting the fitting pin 26 of the retaining member 14 into the fitting holes 63B and 64B of the fluid suppression member 61 and the fitting hole 62A of the nosepiece body 11B, the retaining member 14 can be anchored to the nosepiece body 11B, and the fluid suppression member 61 can be attached to the opening portion 62. Since the position of the fluid suppression member 61 is restricted by the retaining member 14 in the insertion direction Z, the fluid suppression member 61 does not separate from the opening portion 62.

The drape 12 is sandwiched and fixed between the nosepiece body 11B and the retaining member 14. Accordingly, in a case where the tubular portion 16 is inserted into the nose N, the drape 12 is located on the side facing the outside of the body of the patient 1 with respect to the stopper portion 18.

As described above, the drape 12 sandwiched between the nosepiece body 11B and the retaining member 14 protrudes from an outer peripheral edge of the nosepiece body 11B and is provided integrally with the nosepiece body 11B. In addition, similar to the drape 12 of the above first embodiment, it is preferable that the drape 12 is in the folded state when the endoscope nosepiece 60 is not in use, and the folded state is maintained by the pressure-sensitive adhesive tape 15.

After the nosepiece body 11A is worn on the nose N and the mouthpiece body 44 is worn on the mouth M, the doctor peels off the pressure-sensitive adhesive tape 15. By peeling off the pressure-sensitive adhesive tape 15, the drape 42 can be brought to a spread state thereof from the folded state. Then, by mounting the nosepiece body 11A on the nose N, mounting the mouthpiece body 44 on the mouth M, and bringing the drape 42 into the spread state, the head H of the patient 1 can be covered and hidden with the drape 12.

After the nosepiece body 11B is mounted on the nose N and the head H of the patient 1 is covered and hidden with the drape 12, the doctor inserts the insertion part 3 of the endoscope 2 through the pipe line 19 of the endoscope nosepiece 60 into the body of the patient 1 in the insertion direction Z through the nose N. Similar to the endoscope nosepiece 10 of the above first embodiment, since the drape 12 covers and hides the head H of the patient 1, the drape 12 prevents the diffusion of the droplets discharged from the mouth M of the patient 1.

Additionally, the insertion part 3 is inserted into the pipe line 19 through the fluid suppression member 61. However, since the first slit 63A and the second slit 64A intersect each other as described above, the positions of the gaps 63G and 64G do not overlap each other. That is, even in a case where the insertion part 3 is inserted through the fluid suppression member 61, or even in a case where the insertion part 3 is not inserted, the passage of the fluid can be suppressed, and the droplets are prevented from leaking from the fluid suppression member 61.

Additionally, similar to the above first embodiment, the endoscope nosepiece 60 can be removed from the nose of the patient 1 and then be wrapped in the drape 12 and discarded. Therefore, the adhering droplets of the patient 1 can be discarded without being diffused.

In addition, a configuration comprising the mouthpiece 41 similar to the endoscope nosepiece 40 in the above second embodiment in addition to the configuration of the endoscope nosepiece 60 in the above third embodiment may be provided.

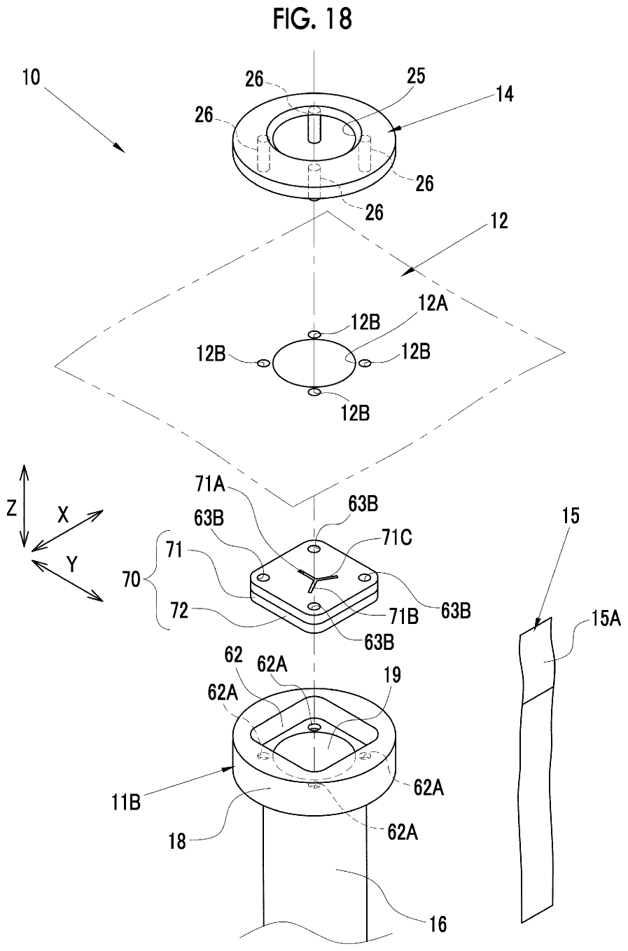

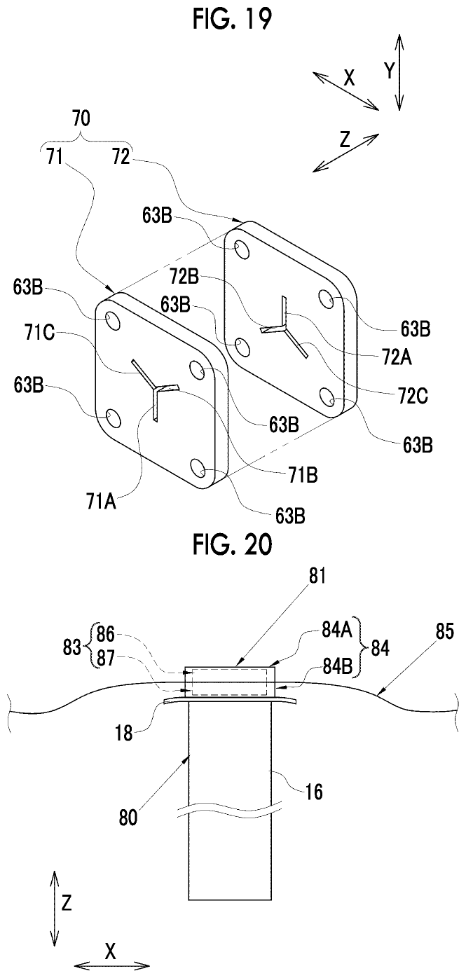

In the above third embodiment, the first porous member 63 and the second porous member 64 constituting the fluid suppression member 61 are each formed with one slit. However, the present invention is not limited to this, and, for example, each porous member may be provided with three or more slits. In a modification example shown in FIGS. 18 and 19, a fluid suppression member 70 includes a first porous member 71 and a second porous member 72, and three slits are disposed in a Y shape.

As shown in FIG. 18, the fluid suppression member 70 is provided in the opening portion 62 of the nosepiece body 11B similarly to the fluid suppression member 61 of the above third embodiment. The fluid suppression member 70 has a first porous member 71 and a second porous member 72.

As shown in FIG. 19, the first porous member 71 is formed by forming a flexible porous material into a rectangular plate shape. The first porous member 71 has three first slits 71A to 71C. The first slits 71A to 71C are disposed in a Y shape. More specifically, the first slits 71A to 71C are disposed at equal angle intervals of 120° and are connected to each other at the center of the first porous member 71. One first slit 71A of the first slits 71A to 71C is perpendicular to one side of the first porous member 71, that is, perpendicular to the left-right direction X, and is disposed parallel to the front-back direction Y. Additionally, the first porous member 71 is formed with a fitting hole 63B into which the fitting pin 26 is fitted, similarly to the first porous member 63 of the above third embodiment.

The second porous member 72 is formed by forming a flexible porous material into a rectangular plate shape. The second porous member 72 has three second slits 72A to 72C. Specifically, the disposition of the second porous member 72 is the same as the first porous member 71 rotated by 180° around a central axis parallel to the insertion direction Z, and similar to the first slits 71A to 71C, the second slits 72A to 72C are disposed at equal angle intervals of 120° and are connected to each other at the center of the second porous member 72. Accordingly, the second slits 72A to 72C are disposed at positions rotated by 180° around the central axis parallel to the insertion direction Z with respect to the first slits 71A to 71C. One second slit 72A of the second slits 72A to 72C is perpendicular to one side of the second porous member 72, that is, perpendicular to the left-right direction X, and is disposed parallel to the front-back direction Y. Cost reduction can be achieved by using two of the same porous members. Additionally, the porous material forming the first porous member 71 and the second porous member 72 is the same as that forming the porous member in each of the above embodiments.

In the fluid suppression member 70, the first porous member 71 and the second porous member 72 are housed in the opening portion 62, and the positions thereof in the insertion direction Z are restricted by the retaining member 14. Accordingly, the first porous member 71 and the second porous member 72 are attached to the opening portion 62 in a laminated state. In addition, although not limited to this, as the fluid suppression member 70, the first porous member 71 and the second porous member 72 may be integrally formed, and the integrally formed fluid suppression member 70 may be attached to the opening portion 62 of the nosepiece body 11B. Additionally, the fluid suppression member 70 may be fixed to the nosepiece body 11B or the like by adhesion without using the retaining member 14.

By providing the above-described fluid suppression member 70 in the nosepiece body 11B, in a case where the insertion part 3 is inserted into the pipe line 19 through the fluid suppression member 70, the insertion part 3 moves in the insertion direction Z while the outer peripheral surface of the insertion part 3 is in close contact with the first slits 71A to 71C and the second slits 72A to 72C. In this case, by providing the three first slits 71A to 71C and the three second slits 72A to 72C in the first porous member 71 and the second porous member 72, respectively, it is possible to insert the insertion part 3 with a resistance smaller than that of the fluid suppression member 61 of the above embodiment in which the slits 63A and 64A are provided one by one. Moreover, it is possible for insertion parts 3 having a small diameter of about 3 mm up to a large diameter of about 16 mm to be inserted with little resistance without any gaps. Accordingly, it is possible to form the first porous member 71 and the second porous member 72 from a porous material having a high density (having few voids), and the passage of the droplets can be further suppressed.

Additionally, since the fluid suppression member 61 of the above embodiment in which the slits 63A and 64A are provided one by one deviates from the position of the slit 64A of the second porous member 64 in a case where the center of the first porous member 63 is not aimed at in a case where the insertion part 3 is inserted, the insertion resistance increases. However, in the case of the fluid suppression member 70, the three first slits 71A to 71C and the second slits 72A to 72C are guided to the center of the first porous member 71 and of the second porous member 72. Therefore, it is not necessary to aim at the center in a case where the insertion part 3 is inserted.

In the fluid suppression member 70 of the modification example shown in FIGS. 18 and 19, three slits are provided in each of the first porous member 71 and the second porous member 72. However, the present invention is not limited to this, and four or more slits may be provided. In addition, even in a case where four or more slits are provided, it is preferable that these slits are disposed at equal angle intervals and are connected to each other at the centers of the first porous member 71 and the second porous member 72.

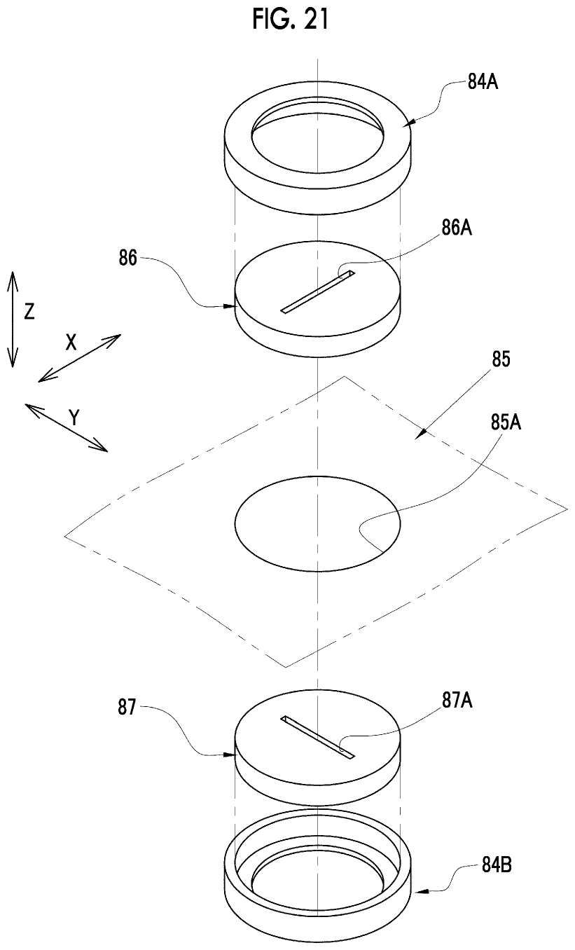

In each of the above embodiments, in a case where the nosepiece body 11A or 11B and the drape 12 or 42 are integrally provided and mounted on the nose of the patient, the drape 12 or 42 is configured to prevent the diffusion of the droplets. However, the present invention is not limited to this, and a configuration may be adopted in which a drape adapter separate from the endoscope nosepiece is provided and the drape adapter is anchored to the endoscope nosepiece. As shown in FIG. 20, a drape adapter 81 is used by being anchored to an endoscope nosepiece 80. The configuration of the endoscope nosepiece 80 is the same as that of the mouthpiece body 11A of the above first and second embodiments, and the configurations of the respective parts will be designated by the same reference numerals, and the description thereof will be omitted.

The drape adapter 81 includes a fluid suppression member 83, a frame member 84, and a drape 85. The fluid suppression member 83 has a first porous member 86 and a second porous member 87. As shown in FIG. 21, the first porous member 86 is formed in a disk shape and has a first slit 86A. The first slit 86A is formed parallel to the left-right direction X and parallel to the insertion direction Z.

The second porous member 87 is formed in a disk shape and has a second slit 87A. The second slit 87A is formed in a direction parallel to the insertion direction Z and intersecting the first slit 86A. More specifically, the second slit 87A is formed parallel to the insertion direction Z and parallel to the front-back direction Y.

The frame member 84 includes a first frame member 84A and a second frame member 84B. The first frame member 84A and the second frame member 84B are fitted to outer peripheral surfaces of the first porous member 86 and the second porous member 87 and sandwich the first porous member 86 and the second porous member 87 in the insertion direction Z.

Similar to the drape 12 or 42 of each of the above embodiments, the drape 85 has a small thickness and is formed in the shape of a quadrangular sheet, for example, is formed of a transparent vinyl sheet. The drape 85 is formed with an opening portion 85A that matches the outer shape of the fluid suppression member 83. Accordingly, since the drape 85 does not block the fluid suppression member 83, the insertion part 3 can be inserted into the pipe line 19 through the fluid suppression member 83. In addition, for convenience of illustration, the drape 85 in FIG. 21 is shown by cutting out only the periphery of the opening portion 85A (a range surrounded by a two-dot chain line), and the actual size thereof is larger than the range surrounded by the two-dot chain line.

The first frame member 84A and the second frame member 84B sandwich the drape 85 in a state of being fitted to the outer peripheral surfaces of the first porous member 86 and the second porous member 87 (a state shown in FIG. 20). Accordingly, the first porous member 86 and the second porous member 87 are integrally provided in a laminated state, and the drape 85 is also integrally provided. In this case, for example, the first frame member 84A and the second frame member 84B are anchored to each other by adhesion or pressure-bonding. In addition, although not limited to this, a fitting pin may be formed in one of the first frame member 84A and the second frame member 84B, and a fitting hole may be formed in the other, and the frame members may be anchored by fitting the fitting pin and the fitting hole to each other.

In addition, the first porous member 86 and the second porous member 87 constituting the fluid suppression member 83 are attached to the frame member 84 in the laminated state. However, the present invention is not limited to this, and the first porous member 86 and the second porous member 87 may be integrally formed as the fluid suppression member 83, and the integrally formed fluid suppression member 83 may be attached to the frame member 84. Alternatively, the drape 85 may be directly anchored to an outer peripheral surface of the integrally formed fluid suppression member 83 without providing the frame member 84.

The drape adapter 81 is anchored to the endoscope nosepiece 80 via, for example, double-sided tape. In this case, it is preferable to anchor the frame member 84 to the endoscope nosepiece 80. In a case where the tubular portion 16 is inserted into the nose N, the fluid suppression member 83 is disposed at an end of the pipe line 19 on the side located outside the body of the patient 1. In a case where the tubular portion 16 is held by the mouth M, the drape 85 is located on the side facing the outside of the body of the patient 1 with respect to the stopper portion 18.

After the endoscope nosepiece 80 is mounted on the nose N of the patient 1, the doctor anchors the drape adapter 81 to the endoscope nosepiece 80. By anchoring the drape adapter 81 to the endoscope nosepiece 80 and bringing the drape 85 in a spread state, the head H of the patient 1 can be covered and hidden with the drape 85.

As described above, after the endoscope nosepiece 80 is mounted on the nose N and the head H of the patient 1 is covered and hidden with the drape 85, the doctor inserts the insertion part 3 of the endoscope 2 through the pipe line 19 of the endoscope nosepiece 80 into the body of the patient 1 through the nose N. Even in a case where the patient 1 coughs or sneezes in a case where the insertion part 3 is inserted through the nose N, the drape 85 covers and hides the head H of the patient 1. Therefore, diffusion of the droplets discharged from the mouth M of the patient 1 can be prevented by the drape 85. Additionally, similar to the above third embodiment, the fluid suppression member 83 can suppress the passage of the fluid, and the droplets can be prevented from leaking from the fluid suppression member 83.

Additionally, the endoscope nosepiece 80 is a disposable type in which the insertion part 3 is removed from the nose N of the patient 1 and is then removed from the nose N of the patient 1 together with the drape 85 and discarded. Accordingly, similar to the above first and second embodiments, since the endoscope nosepiece 80 can be removed from the nose N of the patient 1 and then be wrapped in the drape 85 and discarded, the adhering droplets of the patient 1 are not diffused, and the endoscope nosepiece 80 can be discarded.

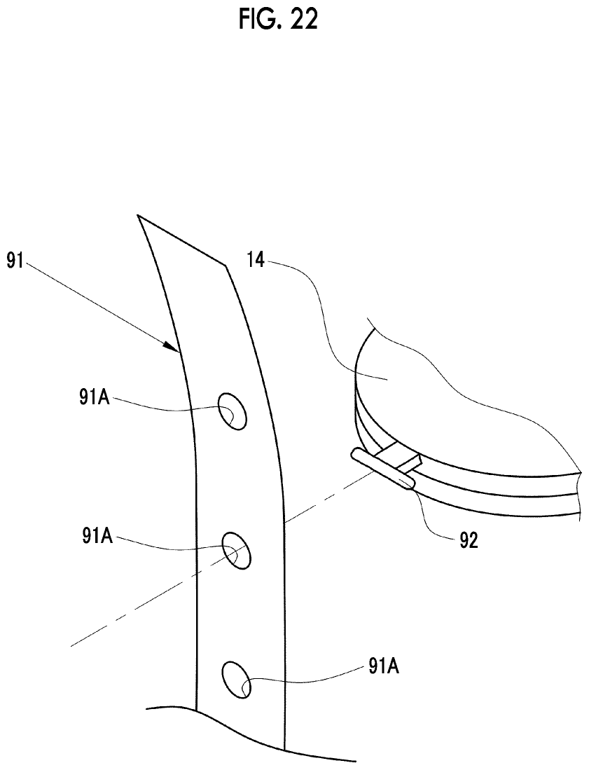

Additionally, as shown in FIG. 22, as another modification example, a pair of band attachment parts 92 may be integrally provided on the nosepiece body 11A, 11B or the retaining member 14, in addition to the configuration of each of the above embodiments. A plurality of mounting holes 91A are formed at an end part of the fixing band 91. The fixing band 91 is formed of, for example, a soft material such as rubber, and the end part of the fixing band 91 can be attached to the mounting holes 91A through the band attachment part 92.

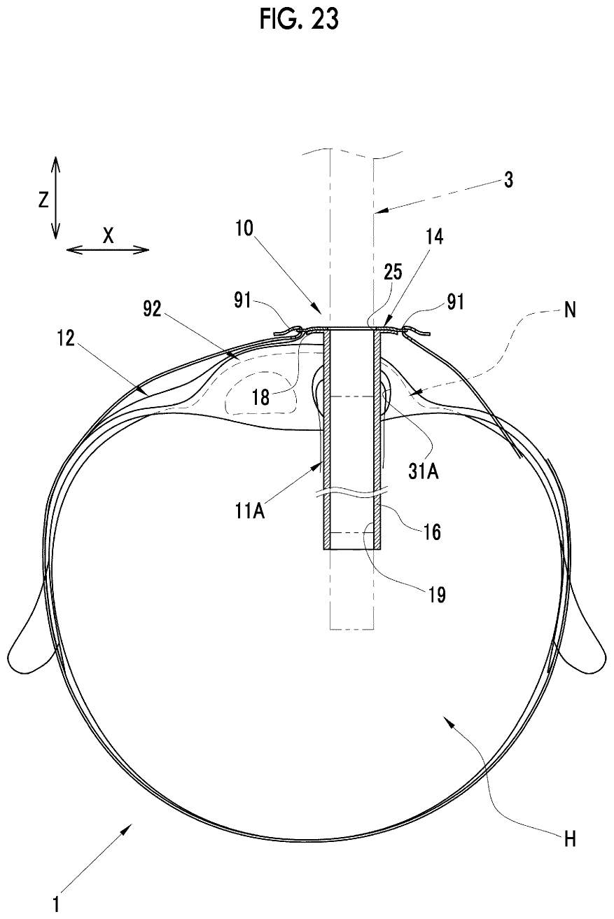

As shown in FIG. 23, by attaching both end parts of the fixing band 91 to the band attachment part 92, the fixing band 91 can be wound around the head H of the patient 1 to fix the endoscope nosepiece 10. Accordingly, the endoscope nosepiece 10 can be reliably worn on the nose N. By changing the positions of the mounting holes 91A through which the band attachment part 92 is passed, the length of the fixing band 91 wound around the head H of the patient 1 can be adjusted.

In each of the above embodiments, examples in which the drapes 12, 42, and 85 are formed from vinyl sheets are given. However, the present invention is not limited to this, and any sheet-like member that covers and hides at least a part of the subject may be adopted. For example, the drapes 12, 42, and 85 may be formed from paper, cloth, or the like. Additionally, in each of the above embodiments, an upper gastrointestinal endoscope is used as an example of the endoscope. However, the present invention is not limited to this, and any nasal type endoscope may be used, and, for example, a bronchoscope may be adopted.

### EXPLANATION OF REFERENCES

1: patient
2: endoscope
3: insertion part
4: operating part
5: universal cord
10: endoscope nosepiece
11A, 11B: nosepiece body
12: drape
12A, 12B: opening portion
14: retaining member
15: pressure-sensitive adhesive tape
15A: end part
16: tubular portion
18: stopper portion
19: pipe line
21: fitting hole
25: opening portion
26: fitting pin
31: nose mask
31A: opening
40: endoscope nosepiece
41: mouthpiece
42: drape
42A: opening portion
43: sponge member
43A: fitting hole
44: mouthpiece body
45: retaining member
46: tubular portion
47: frame portion
48: pipe line
49: opening portion
49A: fitting hole
51: opening portion
52: fitting pin
60: endoscope nosepiece
61: fluid suppression member
62: opening portion
62A: fitting hole
63: first porous member
63A: first slit
63B: fitting hole
63G: gap
64: second porous member
64A: second slit
64B: fitting hole
64G: gap
70: fluid suppression member
71: first porous member
71A: first slit
71B: first slit
71C: first slit
72: second porous member
72A: second slit
72B: second slit
72C: second slit
80: endoscope nosepiece
81: drape adapter
83: fluid suppression member
84: frame member
84A: first frame member
84B: second frame member
85: drape
85A: opening portion
86: first porous member
86A: first slit
87: second porous member
87A: second slit
91: fixing band
91A: mounting hole
92: band attachment part
H: head
L1: length
M: mouth
N: nose
X: left-right direction
Y: front-back direction
Z: insertion direction

What is claimed is:

1. An endoscope nosepiece comprising:
a nosepiece body that is inserted into an external nostril of a subject in a case where an insertion part of an endoscope is inserted into the subject through a nose and that has a pipe line through which the insertion part is inserted; and
a drape that protrudes from an outer peripheral edge of the nosepiece body and that is provided integrally with the nosepiece body to cover at least a part of the subject.

2. The endoscope nosepiece according to claim 1,
wherein the drape is formed in a shape of a sheet and is in a folded state when not in use, and
a holding member is stuck on the drape in the folded state to hold the drape in the folded state.

3. The endoscope nosepiece according to claim 2,
wherein the holding member is at least one of a pressure-sensitive adhesive tape, a locking member, a constraint member, or a coating member.

4. The endoscope nosepiece according to claim 1, further comprising:
a mouthpiece that is held by a mouth of the subject and that is provided integrally with the drape,
wherein the drape protrudes from an outer peripheral edge of the mouthpiece.

5. The endoscope nosepiece according to claim 1,
wherein the nosepiece body has an opening portion provided at an end of the pipe line on a side located outside a body of the subject in a case where the nosepiece body is inserted into the external nostril of the subject, and
a fluid suppression member is provided in the opening portion to suppress passage of a fluid in the pipe line.

6. The endoscope nosepiece according to claim 5,
wherein the fluid suppression member includes:
a first porous member having a first slit parallel to an insertion direction of the insertion part formed therein; and
a second porous member having a second slit parallel to the insertion direction of the insertion part and intersecting the first slit formed therein, and
the fluid suppression member is attached to the opening portion.

7. The endoscope nosepiece according to claim 5,
wherein the fluid suppression member includes:
a first porous member having at least three first slits formed therein; and
a second porous member having at least three second slits formed therein, and
the fluid suppression member is attached to the opening portion.

8. The endoscope nosepiece according to claim 7,
wherein three first slits are formed in the first porous member, and
three second slits are formed in the second porous member.

9. The endoscope nosepiece according to claim 8,
wherein the first porous member and the second porous member have a rectangular outer shape,
one of the first slits is disposed to be perpendicular to one side of the first porous member,
one of the second slits is disposed to be perpendicular to one side of the second porous member, and
the first porous member and the second porous member are attached to the opening portion in a state in which the second slit is disposed at a position rotated by 180° around a central axis parallel to the insertion direction with respect to the first slit.

10. The endoscope nosepiece according to claim 6,
wherein the fluid suppression member is attached to the opening portion in a state in which the first porous member and the second porous member are laminated.

11. An endoscope nosepiece disposal method using an endoscope nosepiece including a nosepiece body that is inserted into an external nostril of a subject in a case where an insertion part of an endoscope is inserted into the subject through a nose, and a drape that is provided integrally with the nosepiece body and that covers at least a part of the subject, the disposal method comprising:
discarding the endoscope nosepiece by removing the insertion part from the subject and then removing the endoscope nosepiece from the nose of the subject together with the drape.

12. An endoscope nosepiece disposal method using an endoscope nosepiece that is inserted into an external nostril of a subject in a case where an insertion part of an endoscope is inserted into the subject through a nose, and a drape that is provided separately from the endoscope nosepiece and that covers at least a part of the subject, the disposal method comprising:
discarding the endoscope nosepiece by removing the insertion part from the subject and then removing the endoscope nosepiece from the nose of the subject together with the drape.
